SEQUENCE LISTING (9 sequences)

SEQ ID NO: 1 - DNA, 747 bp, Homo sapiens
atgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctgggtcaccaggaccaaagggccaaaaaggagaccctggaaaaagtccggatggtgatagtagcctggctgcctcagaaagaaaagctctgcaaacagaaatggcacgtatcaaaaagtggctcaccttctctctgggcaaacaagttgggaacaagttcttcctgaccaatggtgaaataatgacctttgaaaaagtgaaggccttgtgtgtcaagttccaggcctctgtggccacccccaggaatgctgcagagaatggagccattcagaatctcatcaaggaggaagccttcctgggcatcactgatgagaagacagaagggcagtttgtggatctgacaggaaatagactgacctacacaaactggaacgagggtgaacccaacaatgctggttctgatgaagattgtgtattgctactgaaaaatggccagtggaatgacgtcccctgctccacctcccatctggccgtctgtgagttccctatctga

SEQ ID NO: 2 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - DNA, 942 bp, Homo sapiens
atggagctggacagagctgtgggggtcctgggcgctgccaccctgctgctctctttcctgggcatggcctgggctctccaggcggcagacacctgtccagaggtgaagatggtgggcctggagggctctgacaagctcaccattctccgaggctgtccggggctgcctggggcccctgggcccaagggagaggcaggcaccaatggaaagagaggagaacgtggcccccctggacctcctgggaaggcaggaccacctgggcccaacggagcacctggggagccccagccgtgcctgacaggcccgcgtacctgcaaggacctgctagaccgagggcacttcctgagcggctggcacaccatctacctgcccgactgccggcccctgactgtgctctgtgacatggacacggacggagggggctggaccgttttccagcggagggtggatggctctgtggacttctaccgggactgggccacgtacaagcagggcttcggcagtcggctgggggagttctggctggggaatgacaacatccacgccctgaccgcccagggaaccagcgagctccgtgtagacctggtggactttgaggacaactaccagtttgctaagtacagatcattcaaggtggccgacgaggcggagaagtacaatctggtcctgggggccttcgtggagggcagtgcgggagattccctgacgttccacaacaaccagtccttctccaccaaagaccaggacaatgatcttaacaccggaaattgtgctgtgatgtttcagggagcttggtggtacaaaaactgccatgtgtcaaacctgaatggtcgctacctcagggggactcatggcagctttgcaaatggcatcaactggaagtcggggaaaggatacaattatagctacaaggtgtcagagatgaaggtgcgacctgcctag

SEQ ID NO: 4 - protein, 313 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 5 - protein, 251 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 255 aa, Homo sapiens
MELDRAVGVLGAATLLLSFLGMAWALQAADTCPEVKMVGLEGSDKLTILRGCPGLPGAPGPKGEAGTNGKRGERGPPGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 254 aa, Homo sapiens
MELDRAVGVLGAATLLLSFLGMAWALQAADTCPEVKMVGLEGSDKLTILRGCPGLPGAPGPKGEAGTNGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - DNA, 900 bp, Homo sapiens
atggatctactgtggatcctgccctccctgtggcttctcctgcttggggggcctgcctgcctgaagacccaggaacaccccagctgcccaggacccagggaactggaagccagcaaagttgtcctcctgcccagttgtcccggagctccaggaagtcctggggagaagggagccccaggtcctcaagggccacctggaccaccaggcaagatgggccccaagggtgagccaggagatccagtgaacctgctccggtgccaggaaggccccagaaactgccgggagctgttgagccagggcgccaccttgagcggctggtaccatctgtgcctacctgagggcagggccctcccagtcttttgtgacatggacaccgaggggggcggctggctggtgtttcagaggcgccaggatggttctgtggatttcttccgctcttggtcctcctacagagcaggttttgggaaccaagagtctgaattctggctgggaaatgagaatttgcaccagcttactctccagggtaactgggagctgcgggtagagctggaagactttaatggtaaccgtactttcgcccactatgcgaccttccgcctcctcggtgaggtagaccactaccagctggcactgggcaagttctcagagggcactgcaggggattccctgagcctccacagtgggaggccctttaccacctatgacgctgaccacgattcaagcaacagcaactgtgcagtgattgtccacggtgcctggtggtatgcatcctgttaccgatcaaatctcaatggtcgctatgcagtgtctgaggctgccgcccacaaatatggcattgactgggcctcaggccgtggtgtgggccacccctaccgcagggttcggatgatgcttcgatag

SEQ ID NO: 9 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

(19) United States
(12) Patent Application Publication
(10) Pub. No.: US 2010/0331240 A1
(43) Pub. Date: Dec. 30, 2010

(54) METHODS FOR PREVENTION AND TREATMENT OF INFECTIONS WITH SUPRAPHYSIOLOGICAL DOSES OF MANNAN-BINDING LECTIN (MBL) AND FICOLIN-MBL FUSION PROTEINS

(75) Inventors: Ian Michelow, Needham, MA (US); Emmett V. Schmidt, Andover, MA (US); Kazue Takahashi, Cambridge (MA)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/863,306

(22) PCT Filed: Jan. 16, 2009
(86) PCT No.: PCT/US09/31189

§ 371 Date: Jul. 16, 2010

Related U.S. Application Data
(60) Provisional application No. 61/022,096, filed on Jan. 18, 2008.

(51) Int. Cl. A61K 38/17; A61P 31/04; A61P 31/12; A61P 31/14

(52) U.S. Cl. 514/2.4; 514/3.7; 514/2.3

(57) ABSTRACT

The present invention provides methods of treatment and/or prevention of infections, for example, viral and bacterial infections, in individuals, wherein the method comprises administering a supraphysiological amount of mannose-binding lectin (MLB) and/or ficolin-MBL fusion protein to an individual afflicted with an infection or at risk of an infection, such as a bacterial or a viral infection. For example, methods for treatment and/or prevention of Ebola virus infection are provided.

### CROSS-REFERENCE TO RELATED APPLICATIONS

This application claims the benefit under 35 U.S.C. §119(e) of U.S. Provisional Application No. 61/022,096, filed on Jan. 18, 2008, the contents of which are herein incorporated by reference in its entirety.

### BACKGROUND OF THE INVENTION

1. Field of the Invention

The present invention pertains to the use of subunits and oligomers of mannan-binding lectin (MBL) and ficolin-MBL fusion proteins for prevention and/or treatment of infections, particularly in subjects who have normal and functional MBL serum levels.

2. Background of the Invention

Infections count for a large part of morbidity and mortality in the world. While bacterial infections have been tackled by antibiotics and bacteriophages, new treatment methods are sorely needed for the growing amount of bacteria that have become resistant to these treatments. Viruses are a difficult target for treatment in humans and other animals because they use animal cells to replicate and spread. While some viral infections can be prevented using vaccination or antibody-based therapies, several serious and lethal viruses remain currently without effective treatment.

One of such lethal virus family is filoviruses. The two most known lethal filoviruses are Ebola and Marburg viruses. Ebola and Marburg virus can cause acute, lethal hemorrhagic fevers for which no vaccines or effective treatments currently exist. Marburg and Ebola envelope glycoproteins consist of glycoprotein 1 (GP1) and membrane-bound glycoprotein 2 (GP2) protein that are covalently linked by a disulfide bond (Sanchez et al., Proc Natl Acad Sci USA 93:3602-3607, 1996). Although the causes of filovirus virulence are not well known, there is evidence that glycans on the viral glycoproteins play distinct roles in pathogenesis of these viruses (Takeda and Kawaoke, Trends Microbiol 9:506-511, 2001).

It would be useful to discover and develop new treatments for infections, such as viral and bacterial infections that could be used in prevention and/or treatment of infections and/or to supplement the currently available treatment methods to combat infections. In addition, it would be useful to discover new treatments for infectious diseases that do not currently have an effective treatment method, such as filovirus infection or infections by bacteria that have developed resistance to the available antibiotics.

### SUMMARY OF THE INVENTION

The present invention is directed to methods of treatment and/or prevention of infections, for example, viral and bacterial infections, in individuals, wherein the method comprises administering a supraphysiological amount of mannose-binding lectin (MLB) or ficolin-MBL fusion protein to an individual afflicted with an infection or at risk of infection, such as a viral or bacterial infection.

The invention is based upon a surprising discovery, that an infection in an individual with normal MBL serum concentration and function, i.e., who has no defect in MBL, can be successfully treated or prevented by using supraphysiological amounts of MBL or by using ficolin-MBL fusion protein.

The terms “supraphysiological” or “supraphysiologic” are intended to encompass amounts of MBL or ficolin-MBL fusion protein that exceed the normal serum concentration of MBL in an individual, preferably a human individual. The normal serum concentration of MBL can be either measured individually, or estimated based upon a normal range or average normal serum concentration in humans or particular human populations. Typically, the “normal” human serum concentration of MBL is considered a concentration in individuals who do not carry genetic alterations or mutations that are known to reduce the amount or function of MBL in said individual.

In one embodiment, and all other embodiments described herein, one uses amounts of MBL that result in blood concentration of >2× to 10× the average human serum concentration, which is considered a normal serum concentration. In one embodiment, the human average MBL serum concentration is estimated to be about 2 μg/mL. Accordingly, one can use any amount that results in serum concentration of between 4-20 μg/mL. For example, an amount that results in serum concentration of about 4, 5, 6, 7, 8, 9, 10, 11, 12, 13, 14, 15, 16, 17, 18, 19, or 20 μg/mL In one embodiment, similar amounts of ficolin-MBL are used.

Viral infections that can be prevented, ameliorated/treated or cured using the methods of the present invention include, but are not limited to, filoviruses, including Ebola and Marburg viruses, HIV, influenza, severe acute respiratory syndrome coronavirus (SARS-CoV), hepatitis B virus, hepatitis C virus, respiratory syncytial virus, and herpes simplex virus.

Bacterial infections that can be prevented ameliorated/treated or cured using the methods of the present invention include, but are not limited to Staphylococcus aureus; Neisseria meningitidis; Burkholderia multivorans, group B. streptococcus, Escherichia coli, Pseudomonas aeruginosa, Mycoplasma pneumoniae, and Chlamydia pneumoniae.

While the methods of the invention can be used for treatment and/or prevention of infections in any animal or bird, a preferred target individual is human.

In one embodiment, and all other embodiments described herein, the target individual is affected with a bacterium that has become resistant to currently available antibiotics. In one embodiment, one uses the method of the present invention in combination with antibiotics or bacteriophages, or anti-viral agents.

In one embodiment, and all other embodiments described herein, the individual is affected with a filovirus, such as Ebola or Marburg virus.

In one embodiment, and all other embodiments described herein, the method comprises first selecting a patient who is infected with a virus or bacterium, and then administering to the selected individual a supraphysiological amount of MLB or ficolin-MBL fusion protein.

In one embodiment, and all other embodiments described herein, the individual affected with, exposed to or susceptible to be exposed to an infection, such as bacterial or viral infection does not have a congenital or acquired MBL deficiency. In one embodiment, and all other embodiments described herein, one first determines if the individual has a congenital or acquired MBL deficiency. If the individual does not have such a deficiency, the individual can be administered a supraphysiological amount of MBL or ficolin-MBL fusion protein as a treatment or preventive measure to fight a viral infection or a suspected viral infection or exposure to an environment likely to carry viruses, such as an Ebola virus.

MBL can be purified from natural sources or from material produced by recombinant technologies, or by any other suitable MBL-producing cell line, for the prophylaxis and/or treatment of infections. Preparations and pharmaceutical compositions of MBL are known. In one embodiment, one uses the MBL as described in U.S. Pat. No. 5,270,199, which is herein incorporated by reference in its entirety. Also preparations and pharmaceutical compositions of ficolin-MBL fusion proteins are known. In one embodiment, one uses ficolin-MBL chimeric proteins described in, e.g., U.S. Patent Application Publication No. 20060188963. In one embodiment, one uses SEQ ID NO: 1 to produce MBL. Ficolin sequences, for example SEQ ID NO: 3, and SEQ ID NO: 8 can be used to make constructs for recombinantly producing various ficolin-MBL fusion proteins.

One aspect of the invention relates to treatment and/or prophylaxis of infections in individuals affected with a viral or bacterial infection using supraphysiological amount of MBL or ficolin-MBL fusion protein. In one embodiment, the individuals are not immunocompromised.

Without wishing to be bound by a theory, we believe that MBL exerts its antimicrobial activity mainly through its opsonizing activity (preparation of microorganisms for phagocytosis). This activity is dependent on activation of complement after binding of MBL to the microbial surface and deposition of C4b and C3b on the microorganism. MBL can also promote direct complement-mediated killing of the microorganism through an activation of the terminal lytic pathway of complement and insertion of the membrane attack complex (MAC) in the membrane. Without wishing to be bound by a theory, this mechanism is considered of minor importance. Many microorganisms, such as Gram-positive bacteria, e.g., Streptococcus pneumonia, are resistant to MAC, but can be eliminated by opsonophagocytosis. The inhibition of infection may be mediated by MBL directly neutralizing the pathogen, enhancing uptake by phagocytic cells that eliminate the infection, or by killing the pathogens by activation of the complement protein pathway.

Because the MBL is normally present at physiological amounts in individuals who do not have congenital defects in it or who are not immunocompromised, it was surprising that one can exert a virus dose reducing effect by administering additional, supraphysiological amount of MBL into such an individual.

In another aspect, the present invention relates to the use of a composition comprising at least one mannan-binding lectin (MBL) subunit, or at least one oligomer comprising the at least one mannan-binding lectin (MBL) subunit, in the manufacture of a medicament for prophylactic, ameliorating or curative treatment of an infection, including a viral or bacterial infection, in an individual initially having plasma levels of MBL of about 5 μg/mL. In one embodiment, the individual is not genetically disposed to an MBL deficiency or does not have acquired MBL deficiency.

Accordingly, in one embodiment, the methods are used as prophylaxis for individuals who are likely to be exposed, or who have already been exposed to viruses and/or bacteria, but do not yet have symptoms of infection, wherein the presence of supraphysiological amount of MBL or ficolin-MBL will prevent infection or ameliorate symptoms of an infection.

In one embodiment, the invention provides a method of preventing a filovirus infection by administering a supraphysiological dose of MBL or ficolin-MBL fusion protein to an individual who is likely to be exposed to a filovirus. In one embodiment, the filovirus is Ebola virus. In one embodiment, the filovirus is Marburg virus.

In one embodiment, the invention provides use of MBL or ficolin-MBL fusion protein as a medicament for treatment of infections, particularly viral and bacterial infections, in amounts that are supraphysiological.

### BRIEF DESCRIPTION OF DRAWINGS

FIG. 1 shows a schematic drawing of the mannose-binding lectin (MBL) protein and L-ficolin.

FIG. 2 shows a schematic drawing showing the functional and structural domains of MBL and L-ficolin.

FIG. 3 shows a schematic drawing showing the construction of the three chimeric FCN-MBL fusion proteins.

FIG. 4 shows an SDS-PAGE protein gel showing the purified recombinant chimeric FCN-MBL fusion proteins and the denatured purified recombinant MBL under reducing conditions.

FIG. 5 shows a protein gel of the purified recombinant chimeric FCN-MBL fusion proteins and recombinant MBL under non-reducing conditions.

FIG. 6 shows a competitive ELISA comparing avidity of rhMBL and chimeric proteins (100 ng each) binding to mannan.

FIG. 7 shows a C4 deposition assay. The C4 deposition assay is an ELISA-based functional assay that measures the relative capacities of MBL or the chimeric protein to bind human C4. Mannan (10 ug/mL) is coated on a 96-well ELISA plate, blocked with BSA, and incubated with varying concentrations of rhMBL or chimeric proteins. Human C4 (10 ug/mL) is then added and detected with biotin-streptavidin conjugated antibodies. FCN-MBL76 had significantly greater C4 binding activity compared with rhMBL and the other chimerics. This result suggests that FCN-MBL76 has greater complement pathway activating capacity which may result in enhanced pathogen lysis or neutralization.

FIG. 8 shows calreticulin binding assay. The 96-well ELISA plate was coated with rhMBL or chimeric proteins (10 ug/mL), blocked with BSA and incubated with 5 ug/mL biotinylated human placental calreticulin that was measured at absorbance O.D. 405. FCN-MBL76 bound to human placental calreticulin significantly better than rhMBL or the other chimeric proteins. This may have important implications for the relative functions of the proteins because calreticulin is the putative cellular receptor on phagocytes for native MBL and therefore, enhanced binding of the chimeric molecule may result in improved pathogen clearance by opsonophagocytosis.

FIG. 9 shows an inhibition assay using Hep G2 cells infected with lentivirus (HIV) pseudotyped with Ebola glycoprotein. Hep G2 cells at approximately 80% confluence in 96-well tissue culture plates were infected with HIV particles without an envelope (HIV-env neg; solid square) or with an envelope consisting of Ebola glycoprotein (other symbols). The virions encoded luciferase that was expressed only in infected cells and detected with a commercial luciferase assay. Before addition of viral particles to the cells, the viruses were preincubated with 0, 0.1 or 1 ug/mL of rhMBL or chimeric proteins in veronal-buffered saline with 5 mM CaCl2 for 1 hour at 37 C. Infection was achieved by spinoculation of cells at 1000 g×2 hrs. The viral protein mixture was replaced with EMEM culture media and incubated at 37 C for 40 hrs after which, the cells were lyzed and luciferase expression was quantified. rhMBL and the chimeric proteins inhibited viral infection to similar significant extents (1 ug/mL vs no protein, p<0.001)

FIG. 10 shows an inhibition assay using Hep G2 cells infected with native Ebola-Zaire virus. 30,000 Hep G2 cells/well in 96-well tissue culture plates were infected with native Ebola virus (Zaire strain) that was genetically engineered to express GFP. The viral particles were preincubated with 0, 0.1 or 1 ug/mL of rhMBL or chimeric proteins in veronal-buffered saline with 10 mM CaCl2 for 1 hour at 37 C. The viral protein mixture was added to the cells and incubated for 48 hrs after which time the cells were washed. Viral infection of cells was quantified by measuring GFP expression. rhMBL and the chimeric proteins inhibited viral infection but FCN-MBL76 was the most effective.

FIG. 11 shows that a pharmacokinetic modeling of rhMBL (recombinant human MBL) in immunocompetent C57B/6J mice revealed that doses of 75 mcg and 350 mcg doses produced Cmax of ˜5 μg/mL and ˜15 μg/mL, respectively and half-life of ˜11 hours at both doses. A previous study showed that 75 μg is the minimum dose of rhMBL required to activate complement in an MBL-deficient mouse model.

FIG. 12 shows a Kaplan Meier survival analyses: 350 μg rhMBL was given immediately pre-challenge with EBOV Zaire and continued every 12 hrs×10 days resulting in 42% survival rate (log rank, p<0.008).

FIG. 13 shows a Kaplan Meier survival curve with a post-challenge analysis which demonstrated that recombinant human MBL-treated wild-type mice had a significant survival advantage: 40% survived compared to 100% mortality among wild-type and C3 knock-out mice treated with saline or rhMBL indicating that rhMBL provides protection but that the protection is dependent on C3. MBL treated mice survived significantly longer than mice not treated with MBL. EBOV was administered IV 100pfu (plaque forming units) 3000xLD₅₀. WT (wildtype, C57B/6J mice) versus C3 knock out (KO). Recombinant MBL (rhMBL) was administered at 350 mcg IL 12 hors post challenge, then q12hx10 days vs. sham Rx.*log rank, p<0.0004.

FIGS. 14A-14D show that sham treated wild-type mice all died before the 10 day time point. rhMBL-treated wild-type mice had significantly higher total white blood cell and lymphocyte counts after day 5 suggesting that lymphocyte responses in these mice may be protective.

FIG. 15 shows A Rush HepG2 Infection Assay for HIV-EBOZ vs. HIV-env as a negative control with 400 pg/well, 96 well format. MDS (M.R. 1:2, non-HI). The results demonstrate that MBL significantly inhibited infection of HepG2 cells by HIV particles pseudotyped with Ebola glycoprotein. The control virus is an HIV particle without viral surface glycoproteins.

### DETAILED DESCRIPTION OF THE INVENTION

The present invention is directed to methods and uses of MBL and ficolin-MBL fusion proteins for the treatment of infections.

The innate immune system that defends humans from infections is comprised of a network of recognition and effector molecules that act together to protect the host in the first minutes or hours of exposure to an infectious challenge.

The mannan-binding lectin (MBL), synonymous to mannose-binding lectin, mannan-binding protein or mannose-binding protein (MBP), is an evolutionarily conserved circulating host defense protein that acts as a broad spectrum recognition molecule against a wide variety of infectious agents (see, e.g., review by Takahashi et al. Current Opinion in Immunology 18:16-23, 2006).

Several groups of lectins, i.e., carbohydrate-binding proteins, are known in humans. One group is the C-type lectins. The C-type lectins contain a calcium-dependent carbohydrate recognition domain (a C-type CRD)(Weis W I, et al. Immunological Reviews 163: 19-34, 1998). MBL belongs to the subgroup of C-type lectins, termed collectins, since these soluble proteins are composed of subunits presenting three CRDs attached to a collagenous stalk (Holmskov, U., et al., Immunol. Today 15:67-74, 1994). MBL interacts with carbohydrates presented by a wide range of micro-organisms playing an important role in the innate immune defense (Turner, M. W. Immunol. Today 17:532-540, 1996 and Takahashi et al., Current Opinion in Immunology, 18:16-23, 2006). When bound to carbohydrate MBL is able to activate the complement system.

The complement system may be activated via three different pathways: the classical pathway, the alternative pathway, and the third pathway, the mannan-binding lectin (MBL) pathway, which is initiated by the binding of MBL to carbohydrates presented by micro-organisms. The components of the alternative pathway and of the MBL pathway are parts of the innate immune defense, also termed the natural or the non-clonal, immune defense, while the classical pathway involves cooperation with antibodies of the specific immune defense (Janeway C A, Travers P, Walport M and Capra J D, 1999, Immunobiology, the immune system in health and disease, Fourth Edition, Churchill Livingstone).

The human MBL protein is composed of up to 18 identical 32 kDa polypeptide chains (Lu, J., et al., (1990) J. Immunol. 144:2287-2294), each comprising a short N-terminal segment of 21 amino acids including three cysteine residues, followed by 7 repeats of the collagenous motif Gly-X-Y interrupted by a Gln residues followed by another 12 Gly-X-Y repeats. A small 34 residue ‘neck-region’ joins the C-terminal Ca²⁺-dependent lectin domain of 93 amino acids with the collagenous part of the molecule (Sastry, K., et al., (1989) J. Exp. Med. 170:1175-1189).

The collagenous regions of the three polypeptide chains combine to form a subunit which is stabilized covalently by disulphide bridges. Individual subunits are joined by disulphide bridges as well as by non-covalent interactions (Lu, J., et al., J. Immunol. 144:2287-2294, 1990).

The position of these disulphide bridges has, however, not been fully resolved. SDS-PAGE analysis under non-reducing conditions of MBL shows bands with an apparent molecular weight (m.w.) larger than 200 kDa presumably representing blocks of 3, 4, 5 and even 6 assembled subunits (Lu, J., et al., J. Immunol. 144:2287-2294, 1990).

The actual number of subunits in the natural human MBL protein has been controversial. Lipscombe et al. (1995) obtained data by use of ultracentrifugation suggesting 25% of human serum MBL to be made of 2-3 subunits and only a minor fraction reaching the size of 6 subunits (Lipscombe, R. J., et al., Immunology 85:660-667, 1995). The relative quantification was carried out by densitometry of Western blots developed by chemiluminescence (Lu, J., et al., J. Immunol. 144:2287-2294, 1990) found by SDS-PAGE analysis of fractions from ion exchange chromatography that the predominant species of covalently linked MBL subunit chains consisted of tetramers while only pentameric or hexameric complexes activated complement. Gel permeation chromatography (GPC) analysis, in contrast, suggests that MBL is comparable in size with the C1 complex. GPC can be carried out under conditions which allow for a study of the importance of weak protein-protein interactions in the formation of MBL molecules. MBL content in the GPC fractions can be determined by standard MBL assay techniques.

MBL is synthesized in the liver by hepatocytes and secreted into the blood. It binds to carbohydrate structures on bacteria, yeast, parasitic protozoa and viruses, and exhibits antibacterial activity through killing of the microorganisms by activation of the terminal, lytic complement components or through promotion of phagocytosis (opsonization). The sertiform structure of MBL is quite similar to the bouquet-like structure of C1q, the immunoglobulin-binding subcomponent of the first component in the classical pathway (Turner, M. W. Mannose-binding lectin: the pluripotent molecule of the innate immune system. Immunol. Today 17:532-540, 1996). C1q is associated with two serine proteases, C1r and C1s, to form the C1 complex. Similarly, MBL is associated with two serine proteases MASP-1 (Matsushita, M. and Fujita, T, J. Exp. Med. 176:1497-1502, 1992) and MASP-2 (Thiel S, et al., Nature, 386(6624): 506-510, 1997), and an additional protein called Map19 (Stover C M, et al., J Immunol 162: 3481-3490, 1999). MASP-1 and MASP-2 have modular structures identical to those of C1r and C1s (Thiel S, et al., Nature, 386(6624): 506-510, 1997). The binding of MBL to carbohydrates induces the activation of MASP-1 and MASP-2. MASP-2 then generates the C3 convertase, C4b2a, through cleavage of C4 and C2. Reports suggest that MASP-1 may activate C3 directly. Nothing is known about the stoichiometry and activation sequence of the MBL/MASP complexes. MBL has also been characterized in other animals such as rodents, cattle, chicken and monkeys.

Based on presence and function of MBL in at least rodents, cattle, chicken and monkeys, in addition to humans, makes the methods of the present invention applicable to at least these animals as well.

Human mannose-binding protein has been disclosed in U.S. Pat. No. 5,270,199. Moreover, use of MBL in treatment of immunocompromised individuals has been described (U.S. Pat. Nos. 6,562,784 and 7,202,207, and U.S. Patent Application Publication No. 2007-0197428). However, because MBL is a naturally occurring molecule present in the serum, no one has suggested its use in treatment or prevention of infections in individuals with normal serum concentration of MBL. Our discovery that supraphysiological amounts of MBL can increase the infection fighting capacity of an individual with normal MBL concentrations and function was thus surprising.

Accordingly, one aspect of the invention provides a method for prevention and treatment of infections in individuals, such as human individuals, comprising administering to said individual a supraphysiological amount of MBL.

The term “supraphysiological” as used in the present application means amounts greater than the physiological amount normally present in an individual or greater than minimal concentration of MBL required to activate a complement, i.e. to bind to C4. Similar concentrations of ficolin-MBL fusion proteins can also be used.

In one embodiment, one uses MBL and/or ficolin-MBL fusion proteins or combinations thereof in the amount that results in the amount of about 2-10 times greater than the physiological amount of MBL in an individual. In one embodiment, one uses, for example, 2, 3, 4, 5, 6, 7, 8, 9 or 10 times greater than the physiological amount in the individual. In one embodiment, the average physiological amount is considered about 2 μg/mL. In one embodiment, one first determines the physiological amount of MBL in an individual prior to administering MBL or ficolin-MBL fusion protein composition to said individual. This is particularly useful when using MBL or ficolin-MBL as a prophylaxis for individuals who will be at risk of encountering infective agents, such as medical personnel or armed forces who may be a target for a biological attack.

The concentration of MBL in human serum is largely genetically determined, but reportedly increases up to threefold during acute phase infection reactions (Thiel S, et al., Clin Exp Immunol 90: 31-35, 1992). Three mutations causing structural alterations and two mutations in the promotor region are associated with MBL deficiency (Madsen, H. O., et al., Immunogenetics 40:37-44, 1994). MBL deficiency is associated with susceptibility to a variety of infections (Summerfield J A, et al., Lancet 345: 886-889, 1995; Garred P, et al., Lancet 346: 941-943, 1995).

It has been estimated that the average physiological amount of MBL in human serum is about 2 μg/mL. Accordingly, in one embodiment, one uses the average physiological amount as the physiological amount, and consequently, the supraphysiological amount of MBL or ficolin-MBL according to the present invention is about 2-10 times above the average physiological MBL level.

In one embodiment, one takes into account the impact of MBL haplotypes when considering the physiological serum MBL concentrations. At least ten distinct MBL haplotypes have been described in human, four of which (LYPB, LYQC, HYPD and LXPA) dictate low serum MBL concentrations (Madsen et al. J Immunol 161:3169-3175, 1998; Takahashi et al., Current Opinion in Immunology 18:16-23, 2006). Human populations from diverse geographic locations and ethnic and genetic backgrounds have higher rate of haplotype variation, with a rate of heterozygosity from 15% in white populations to 30% in certain African populations. Accordingly, in one embodiment, to establish the physiological serum MBL level to adjust the amount of MBL used in the methods of the present invention, one correlates the level of MBL with a functional measurement of the MBL:MASP pathway (Takahashi et al., Current Opinion in Immunology 18:16-23, 2006; Petersen et al., J Immunol Methods 257:107-116, 2001). Accordingly, if the average serum concentration of MBL in a particular individual or population is higher, the supraphysiologic dosage is adjusted accordingly. Similarly, if the average serum concentration of MBL is lower, a lower amount is needed for the treatment of prevention of infections. A skilled artisan is easily able to make these determinations based on the description herein.

A wide range of oligosaccharides can bind to MBL. As the target sugars are not normally exposed on mammalian cell surfaces at high densities, MBL does not usually recognize self-determinants, but is well suited to interactions with microbial cell surfaces presenting repetitive carbohydrate determinants. In vitro, yeast (Candida albicans and Cryptococcus neoformans), viruses (HIV-1, HIV-2, HSV-2, and various types of influenza A) and a number of bacteria have been shown to be recognized by MBL. In the case of some bacteria, the binding with MBL is impaired by the presence of a capsule (van Emmerik, L C, et al., Clin.Exp.Immunol. 97:411-416, 1994). However, even encapsulated bacteria (Neisseria meningitidis) can show strong binding of MBL (Jack D L, et al., J Immunol 160: 1346-1353, 1998), and is thus one target infection according to the present invention.

The microorganisms, which infect MBL deficient individuals, represent many different species of bacterial, viral and fungal origin (Summerfield J A, et al., BioMed J 314: 1229-1232, 1997; Miller, M. E., et al., Lancet: 60-63, 1968; Super, M., et al., Lancet 2:1236-1239, 1989; and Nielsen, S. L., et al., Clin. Exp. Immunol. 100:219-222, 1995). Deficiency is also associated with habitual abortions (Christiansen, O. B., et al., Scand. J. Immunol., 49, 193-196, 1999). Indeed, MBL appears to be a general defense molecule against most bacteria, and thus be considered as one reason why so many bacteria are non-pathogenic.

Accordingly, in one embodiment, the methods of the invention pertain to prevention and/or treatment of infections caused by any of the foregoing infective agents, including viruses, yeast, fungus, and bacteria.

While accumulating data support the notion of a protective effect of MBL there are also observations suggesting that infections with some microorganisms, notably intracellular pathogens, attain a higher frequency in MBL sufficient than in MBL deficient individuals (Garred, P, et al., Eur. J. Immunogen. 21:125-131, 1994; Hoal-Van Helden E G, et al., Pediatr Res 45:459-64, 1999). This is in concordance with the results of an animal experiment, where an increased number of HSV-2 were found in the liver of mice pre-injected with human MBL (Fischer, P B, et al., Scand J Immunol 39:439-445, 1994). Our results contradict these findings by showing a strong protective and treatment effect of administering to a subject a supraphysiological amount of MBL and/or ficolin-MBL fusion proteins or combinations thereof.

Clinical grade MBL has been obtained from blood donor plasma and shown to be safe upon infusion (Valdimarsson, H., M. et al., Scand. J. Immunol. 48:116-123, 1998). Accordingly, one can use such preparations in the methods of the present invention. Similarly, one can make recombinant MBL using any well known gene expression system.

Ficolins, like MBL, are lectins that contain a collagen-like domain. However, unlike MBL, they have a fibrinogen-like domain, which is similar to fibrinogen beta- and gamma-chains. Ficolin also forms oligomers of structural subunits, each of which is composed of three identical 35 kDa polypeptides. Each subunit is composed of an amino-terminal, cysteine-rich region; a collagen-like domain that consists of tandem repeats of Gly-Xaa-Yaa triplet sequences (where Xaa and Yaa represent any amino acid); a neck region; and a fibrinogen-like domain. The oligomers of ficolins comprise two or more subunits, especially a tetrameric form of ficolin has been observed.

Some of the ficolins trigger an activation of the complement system substantially in similar way as done by MBL. This triggering of the complement system results in the activation of novel serine proteases (MASPs).

The fibrinogen-like domain of several lectins has a similar function to the CRD of C-type lectins including MBL, and function as pattern-recognition receptors to discriminate pathogens from self.

Serum ficolins have a common binding specificity for GlcNAc (N-acetyl-glucosamine), elastin or GalNAc (N-acetyl-galactosamine). The fibrinogen-like domain is responsible for the carbohydrate binding. In human serum, two types of ficolin, known as L-ficolin (also called P35, ficolin L, ficolin 2 or hucolin) and H-ficolin (also called Hakata antigen, ficolin 3 or thermolabile b2-macroglycoprotein), have been identified, and both of them have lectin activity. L-ficolin recognises GlcNAc and H-ficolin recognises GalNAc. Another ficolin known as M-ficolin (also called P35-related protein, ficolin 1 or ficolin A) is not considered to be a serum protein and is found in leucocytes and in the lungs. L-ficolin and H-ficolin activate the lectin-complement pathway in association with MASPs. M-Ficolin, L-ficolin and H-ficolin has calcium-independent lectin activity.

Accordingly, in one embodiment, the invention provides methods of prevention and/or treatment of infectious diseases using MBL-L-ficolin or MBL-H-ficolin fusion proteins, or a combination thereof.

Naturally, one can also use a combination of MBL and MBL-ficolin, such as MBL-L-ficolin and/or MBL-H-ficolin.

Chimeric molecules of MBL and ficolin have been described, for example, in U.S. Patent Application Publication No. 2006-0188963. Although it has been suggested that the chimeric molecules could be used to prevent and/or treat infections in patients having clinical symptoms associated with congenital or acquired MBL deficiency or being at risk of developing such symptoms (Id.), no one has proposed or shown that individuals with normal MBL activity would benefit from additional, supraphysiological amounts of MBL or MBL-ficolin in combating infectious diseases.

Based on our findings, the present invention provides a novel method for treatment or prevention of infections in an individual having normal expression and normal function of MBL.

In addition, fusion proteins useful according to the methods of the invention can designed in such a way as to test whether the source of the MASP-binding site and flanking sequences, and presence of the “kink” from MBL affect ligand binding activity and/or complement activation. Without wishing to be bound by a theory, we designed the proteins in the examples based on the assumption that differences at these sites alter the protein conformational structure which in turn alters protein-protein interactions. Therefore any differences in protein activity can assist in understanding the functional parts of the molecules. We discovered that FCN-MBL76 has a greater activity in various assays. Without wishing to be bound by a theory, we concluded that is because of differences in spatial orientation of the CRDs. We have shown that FCN-MBL76 binds the best to a sugar, mannan.

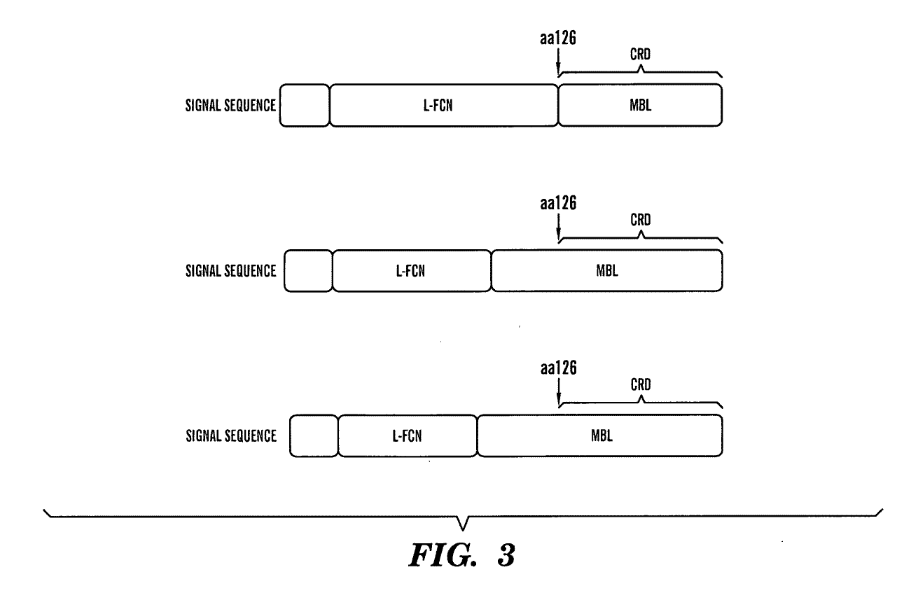

Examples of useful fusion proteins are presented in FIG. 3. In our test molecules, FCN-MBL126 has only the carbohydrate recognition domain (CRD) from MBL and the rest includes the MASP-binding site. The amino-terminus is from L-FCN.

FCN-MBL76 has the CRD, neck and part of the flanking sequences of the MASP-binding site from MBL; the lysine and other flanking sequences of the MASP-binding site, and the amino-terminal is from L-FCN.

FCN-MBL64 has the CRD, neck, MASP-binding site and flanking sequences and the “kink” from MBL; the amino-terminal is from L-FCN.

In one embodiment, one uses a fusion protein which includes the signal peptide from L-FCN because this component is important to signal the protein to be transported from the cytosol to the endoplasmic reticulum for packaging and secretion.

Accordingly, based on the description herein and throughout this specification and examples, a skilled artisan can design various fusion proteins, including proteins with stability-increasing modifications using routine methods.

In certain embodiments, the methods of the present invention include treatment and/or prevention of infections including bacterial, viral and fungal infections. The viral infections according to the present invention can be caused by any virus, such as viruses including but not limited to the viruses of the herpes family, such as Herpes Simpex I, Herpes Simplex II, Human Herpesvirus 6 (HHV-6), herpes zoster; poxviruses; corona viruses; paramyxoviruses; and togaviruses, HIV, Ebola, and the like.

In certain embodiments, the methods of the present invention provide for treatment of bacterial infections and/or preventing bacterial infection for bacteria such as Staphylococcus spp., Streptococcus spp., Escherichia spp., Enterococcus spp., Pseudomonas spp. bacteria and combinations thereof, and more particularly Staphylococcus aureus, including antibiotic resistant strains such as methicillin resistant Staphylococcus aureus, Staphylococcus epidermidis, Escherichia coli (E. coli), Pseudomonas aeruginosa (Pseudomonasae), Streptococcus pyogenes, and combinations thereof.

In certain embodiments, the method of the present invention provide treatment and/or prevention for infections caused by Staphylococcus aureus; Neisseria meningitidis; Burkholderia multivorans, group B streptococcus, Escherichia coli, Pseudomonas aeruginosa, Mycoplasma pneumoniae, and Chlamydia pneumoniae.

In one embodiment, the method of the present invention provide treatment and/or prevention for infections caused by HIV, influenza, severe acute respiratory syndrome coronavirus SARS-CoV), hepatitis B virus, hepatitis C virus, respiratory syncytial virus, herpes simplex virus, or filovirus, for example Ebola or Marburg virus.

A medicament comprising MBL and/or MBL-ficolin fusion protein, may be produced by using the eluant obtained from the affinity chromatography as such. It is however preferred that the eluant is subjected to further purification steps before being used in a pharmaceutically acceptable carrier.

In one embodiment, the composition or medicament consists essentially of MBL and/or MBL-ficolin fusion protein or functional, i.e. infectious agents binding derivatives thereof in a pharmaceutically acceptable carrier.

In addition to the MBL oligomers or ficolin-MBL fusion proteins, the medicament may comprise a pharmaceutically acceptable carrier substance and/or vehicles. In particular, a stabilizing agent may be added to stabilize the MBL proteins or the ficolin-MBL fusion proteins. The stabilizing agent may be a sugar alcohol, saccharides, proteins and/or amino acids. Examples of stabilizing agents are maltose or albumin.

The term “derivative” as used herein refers to MBL or ficolin-MBL fusion proteins which are functional in the sense that they can bind infectious agents but have also have been chemically modified, for example but not limited to by techniques such as ubiquitination, labeling, pegylation (derivatization with polyethylene glycol, PEG) or addition of other molecules. A molecule also a “derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990), and PROTEINS—STRUCTURE AND MOLECULAR PROPERTIES, 2nd Ed., T. E. Creighton, W.H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).).

In one embodiment, the MBL and/or ficolin-MBL fusion protein is fused to a second fusion partner, such as a carrier molecule to enhance its bioavailability. Such carriers are known in the art and include poly (alkyl) glycol such as poly ethylene glycol (PEG). Fusion to serum albumin can also increase the serum half-life of therapeutic polypeptides.

The MBL and/or ficolin MBL fusion polypeptide can also be fused to a second fusion partner, for example, to a polypeptide that targets the product to a desired location, or, for example, a tag that facilitates its purification, if so desired. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

As used herein, the term “conjugate” or “conjugation” refers to the attachment of two or more entities to form one entity. For example, the methods of the present invention provide conjugation of a MBL or ficolin-MBL fusion polypeptide or fragments, derivatives or variants thereof, joined with another entity, for example a moiety such as a first fusion partner that makes the MBL or ficolin-MBL fusion protein stable, such as Ig carrier particle, for example IgG1 Fc. The attachment can be by means of linkers, chemical modification, peptide linkers, chemical linkers, covalent or non-covalent bonds, or protein fusion or by any means known to one skilled in the art. The joining can be permanent or reversible. In some embodiments, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention.

According to the present invention, the MBL or ficolin-MBL fusion polypeptide or fragments, derivatives or variants thereof, can be linked to the first fusion partner via any suitable means, as known in the art, see for example U.S. Pat. Nos. 4,625,014, 5,057,301 and 5, 514,363, which are incorporated herein in their entirety by reference. For example, the MBL or ficolin-MBL fusion polypeptide can be covalently conjugated to the IgG1 Fc, either directly or through one or more linkers. In one embodiment, a MBL or ficolin-MBL fusion polypeptide as disclosed herein is conjugated directly to the first fusion partner (e.g. Fc), and in an alternative embodiment, a MBL or ficolin-MBL fusion polypeptide as disclosed herein can be conjugated to a first fusion partner (such as IgG1 Fc) via a linker, e.g. a transport enhancing linker.

As used herein, the term “treating” includes reducing or alleviating at least one adverse effect or symptom of an infection. Accordingly, the anti-viral medicament according to the present invention may be a medicament capable of virus attenuation and/or elimination. Similarly, antibacterial medicament according to the present invention may be a medicament capable of stabilizing the bacterial infection and/or eliminating such an infection.

The MBL or ficolin-MBL fusion protein can be administered by any appropriate route which results in an effective treatment of an infection in the subject. In one embodiment, the administration is performed systemically.

In one embodiment, one administers the MBL or ficolin-MBL fusion proteins enterally, topically or parenterally. The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and infrasternal injection and infusion. The phrases “systemic administration,” “administered systemically”, “peripheral administration” and “administered peripherally” as used herein mean the administration of MBL and/or ficolin-MBL fusion protein other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Other conventional additives may be added to the medicament depending on administration form for example. In one embodiment the medicament is in a form suitable for injections. Conventional carrier substances, such as isotonic saline, may be used.

In another embodiment the pharmaceutical composition or medicament is in a form suitable for pulmonal administration, such as in the form of a powder for inhalation or cream or fluid for topical application.

The route of administration may be any suitable route, such as intravenously, intramusculary, intraperitoneally, subcutanously or intradermally. Also, pulmonal or topical administration is envisaged by the present invention.

The MBL composition may also be administered simultaneously, sequentially or separately with another anti-bacterial, anti-viral or viral or bacterial infection symptom alleviating treatment.

The MBL and/or ficolin-MBL composition is administered in suitable dosage regimes, in particular, it is administered repeatedly at suitable intervals, such as once or twice a week. For example, one can start before exposure to the virus and maintain the periodic administration at intervals, for example 1, 2, 3, 4, 5, 6, or 7 times a week, or, for example, 1, 2, 3, or 4 times a day, at least during a part of the exposure or suspected exposure of the individual to the virus. One can also begin administering the MBL composition at the time of suspected exposure and continue with periodic additional dosages for at least 2, 3, 4, 5, 6, or 7 days or even longer. One can also begin the treatment at the onset of the symptoms of the infection, such a viral infection and continue the periodic administration of at least one additional dosages until the symptoms begin to diminish or until there are no symptoms, or until at least 1, 2, 3, 4, 5, 6, 7 days after the symptoms have disappeared.

In one embodiment, the invention provides a method wherein recombinant human MBL is administered every 1, 2, 3, 4, 5, 6, 7, 8, 9, 10 11, 12, 13, 14, 15, 16, 17, 18, 19, 20, 21, 22, 23, or 24 hours. In one embodiment, one administers recombinant human MBL every 12 hours.

The use of an MBL or ficolin-MBL fusion protein composition may also be in a kit-of-parts further comprising another medicament, such as an anti-fungal, anti-yeast, anti-bacterial and/or anti-viral medicament.

Accordingly, in one embodiment, the invention provides a method for treatment and/or prevention of an infection in an individual comprising administering to said individual a supraphysiological amount of mannose-binding lectin (MBL) or ficolin-MBL fusion protein or a combination thereof and a pharmaceutically acceptable carrier. In one embodiment, the infection is a viral infection. In one embodiment, the viral infection is an Ebola virus infection. In another embodiment, the infection is a bacterial infection.

In one embodiment the supraphysiological amount is an amount that results in blood concentrations of the MBL or the ficolin-MBL fusion protein at about 2-10 times the average physiological MBL serum concentration. In one embodiment, the average physiological MBL serum concentration is about 2 μg/mL.

In one aspect of the invention and all other aspects described herein, the invention provides use of supraphysiological amount of mannose-binding lectin (MBL) or ficolin-MBL fusion protein for treatment and/or prevention of an infection. In one embodiment, the infection is a viral infection. In one embodiment, the viral infection is an Ebola virus infection. In another embodiment, the infection is a bacterial infection.

In one aspect of the invention and all other aspects described herein, the invention provides, one uses MBL2, SEQ ID NO: 2, SEQ ID NO: 5, SEQ ID NO: 6 and/or SEQ ID NO: 7, or combinations thereof in the methods of the invention. A skilled artisan can create alternative protein variants and fusion proteins for the use in the methods of the present invention using routine gene manipulation techniques and sequences available in public and proprietary databases alike.

### EXAMPLES

We have developed novel immunotherapeutic agents that effectively prevent and treat infections, such as life-threatening infections, like infections caused by Ebola (EBOV) or Marburg viruses. In our experiments we used Ebola viruses as examples. However, based on our discovery, any other viral or bacterial infection can be treated using similar methods.

Ebola viruses are filamentous, enveloped, non-segmented, negative-strand RNA viruses. EBOV subspecies Zaire and Sudan are highly pathogenic in humans and cause as high as 90% mortality in outbreaks in equatorial Africa. There are no FDA-approved vaccines or therapeutic agents available to prevent or treat EBOV.

MBL is a broad-spectrum ligand-specific C-type lectin that plays an important role in innate immunity by acting as an opsonin and by activating the lectin complement pathway. Preliminary data indicated that recombinant human MBL (rhMBL) 1) binds high-mannose residues in EBOV envelope glycoproteins (GP) which are the principal virulence and immunogenic determinants of EBOV, and Holmskov, U., et al., Immunol. Today 15:67-74, 1994) inhibits experimental EBOV infection in a complement-dependent manner manner (Ji X et al. J Gen Virol 2005; 86:2535-42). These observations strongly support rhMBL's role as a novel immunotherapeutic agent for EBOV.

We also tested three chimeric proteins (FCN-MBL) comprising varying lengths of the carboxy terminal of MBL and the collagen stalk of L-ficolin, another lectin-like protein that activates the lectin complement pathway (see FIGS. 3 and 3).

RhMBL and FCN-MBL chimeras have comparable ligand-binding specificity but FCN-MBL has a simpler multimeric structure and different functional characteristics, and rhMBL reduces mortality in a mouse model of native EBOV Zaire infection.

Clinical grade rhMBL was provided by ENZON Pharmaceuticals, NJ.

Chimeric FCN-MBL proteins were expressed in stably transfected HEK293F cells cultured in an artificial capillary cell culture system (CELLMAX, Spectrum Laboratories, CA). Plasmids were provided by ENZON Pharmaceuticals, NJ. Proteins were batch purified with mannose-agarose beads, eluted with EDTA-containing buffer, and then dialyzed with the same buffer as for rhMBL.

The designs of the three chimeric FCN-MBL fusion proteins are as follows:

L-FCN-MBL126: L-FCN (signal sequence+collagen+“hinge” to ficolin domain amino acid [aa]128)+MBL (from aa126 carbohydrate binding domain [CRD]) (SEQ ID NO: 5).

L-FCN-MBL76: L-FCN (signal sequence+part of collagen to aa82)+MBL (from aa76 including rest of collagen+coil-coil+carbohydrate binding domain) (SEQ ID NO: 6).

L-FCN-MBL64: L-FCN (signal sequence+part of collagen to aa69)+MBL (from aa64 rest of collagen [containing “kink”]+coil-coil+carbohydrate binding domain) (SEQ ID NO: 7).

Endotoxin assay: endotoxin was <5 EU/mL (FDA standard) in all protein preparations as determined by the kinetic Limulus amebocyte lysate test.

Ebola viruses: For in vitro experiments, HIV particles (env-negative pNL 4-3) that lacked gp120/gp41 and that expressed luciferase were pseudo-typed with Ebola glycoprotein. Viral concentrations were determined by ELISA for p24 core protein. Native EBOV subspecies Zaire was used for mouse experiments.

Structure and function of rhMBL and chimeric proteins: the relative oligomerization was demonstrated with SDS-PAGE; protein composition was determined by amino acid analysis. Relative capacity to activate complement was determined by C4 deposition in an ELISA format with mannan as the capture antigen; and relative avidity was studied with a range of acetylated and non-acetylated carbohydrates in a competitive ELISA format.

HEK293F infection-inhibition assay: 400 pg p24 HIV-Ebola GP particles were preincubated with MBL-deficient serum (1:2 dilution) that was supplemented with varying amounts of rhMBL to test the relative capacity of rhMBL to inhibit infection. HEK293F cells (5×10³/well in a 96-well format) were then infected with the viral particles by means of spinoculation×2 hrs and then incubated with virus-free fresh media×40 hrs. Cell infection was determined by expression of luciferase.

Murine model of EBOV infection: relevant PK parameters (t1/2, Cmax) of rhMBL were calculated in 8-week old C57B/6J mice (n=5 per group) after I.P. injection of 75 mcg (3 mg/kg) or 350 mcg (14 mg/kg). C57B/6J mice were challenged with 100pfu EBOV Zaire (3000×LD50) I.P. immediately after or 12 hours before treatment with rhMBL 350 mcg I.P. that was continued every 12 hours×10 days.

### Results

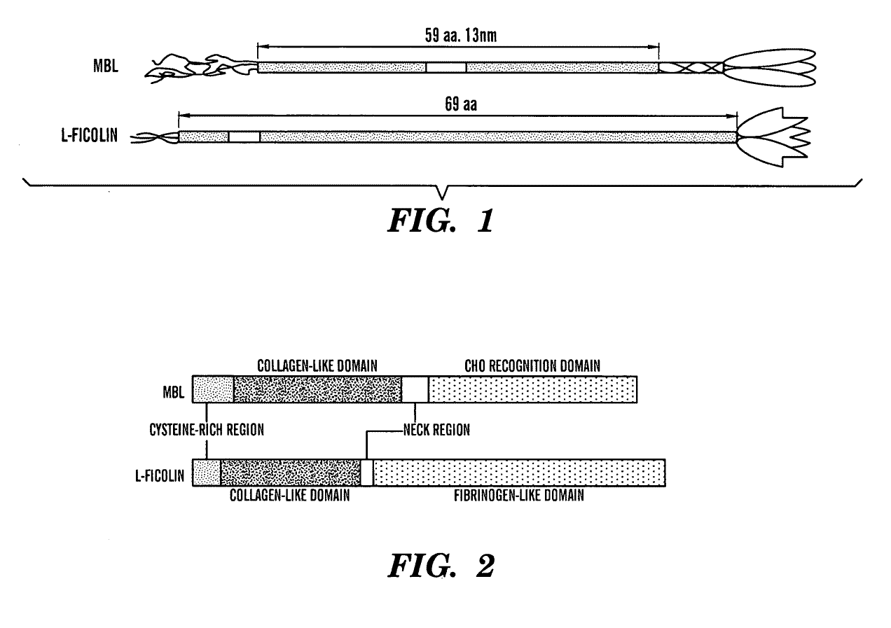

Three chimeric proteins (FCN-MBL) were designed (FIG. 3). The chimeric FCN-MBL proteins comprise varying lengths of the carboxyl terminal of MBL (FIGS. 1 and 2) and the collagen stalk of L-ficolin, another lectin-like protein that activates the lectin complement pathway (FIG. 2). The carboxyl terminal of MBL has been shown to be the region responsible for binding carbohydrates, the carbohydrate recognition domain (CRD) (FIG. 2). Human MBL nucleotide reference sequence, MBL2 CDS was derived from a consensus CDS 7247.1/NCBI NM_{—}000242.2, the sequence of the 747 by nucleic acid is as follows: atgtccctgt ttccatcact ccctctcctt ctcctgagta tggtggcagc gtcttactca gaaactgtga cctgtgagga tgcccaaaag acctgccctg cagtgattgc ctgtagctct ccaggcatca acggcttccc aggcaaagat gggcgtgatg gcaccaaggg agaaaagggg gaaccaggcc aagggctcag aggcttacag ggcccccctg gaaagttggg gcctccagga aatccagggc cttctgggtc accaggacca aagggccaaa aaggagaccc tggaaaaagt ccggatggtg atagtagcct ggctgcctca gaaagaaaag ctctgcaaac agaaatggca cgtatcaaaa agtggctcac cttctctctg ggcaaacaag ttgggaacaa gttcttcctg accaatggtg aaataatgac ctttgaaaaa gtgaaggcct tgtgtgtcaa gttccaggcc tctgtggcca cccccaggaa tgctgcagag aatggagcca ttcagaatct catcaaggag gaagccttcc tgggcatcac tgatgagaag acagaagggc agtttgtgga tctgacagga aatagactga cctacacaaa ctggaacgag ggtgaaccca acaatgctgg ttctgatgaa gattgtgtat tgctactgaa aaatggccag tggaatgacg tcccctgctc cacctcccat ctggccgtct gtgagttccc tatctga (SEQ ID NO: 1).

Human MBL protein reference sequence, translation of MBL2 results in a 248 amino acid sequence as follows: mslfpslpll llsmvaasys etvtcedaqk tcpaviacss pgingfpgkd grdg/kgekg epgqglrglq gppgklgppg npgpsgspgp kgqkgdpgks pdgdsslaas erkalqtema rikkwltfsl gkqvgnkffl tngeimtfek vkalcvkfqa svatprnaae ngaiqnlike eaflgitdek tegqfvdltg nrltytnwne gepnnagsde dCvlllkngq wndvpcstsh lavcefpi (SEQ ID NO: 2).

Human L-ficolin nucleotide reference sequence FCN2 CDS was derived from consensus 6983.1/NCBI NM_{—}004108.2. The sequence of this 942 nucleic acid sequence is as follows:

(SEQ ID NO: 3)
atggagctgg acagagctgt gggggtcctg ggcgctgcca
ccctgctgct ctctttcctg ggcatggcct gggctctcca
ggcggcagac acctgtccag aggtgaagat ggtgggcctg
gagggctctg acaagctcac cattctccga ggctgtccgg
ggctgcctgg ggcccctggg cccaagggag aggcaggcac
caatggaaag agaggagaac gtggcccccc tggacctcct
gggaaggcag gaccacctgg gcccaacgga gcacctgggg
agccccagcc gtgcctgaca ggcccgcgta cctgcaagga
cctgctagac cgagggcact tcctgagcgg ctggcacacc
atctacctgc ccgactgccg gcccctgact gtgctctgtg
acatggacac ggacggaggg ggctggaccg ttttccagcg
gagggtggat ggctctgtgg acttctaccg ggactgggcc
acgtacaagc agggcttcgg cagtcggctg ggggagttct
ggctggggaa tgacaacatc cacgccctga ccgcccaggg
aaccagcgag ctccgtgtag acctggtgga ctttgaggac
aactaccagt ttgctaagta cagatcattc aaggtggccg
acgaggcgga gaagtacaat ctggtcctgg gggccttcgt
ggagggcagt gcgggagatt ccctgacgtt ccacaacaac
cagtccttct ccaccaaaga ccaggacaat gatcttaaca
ccggaaattg tgctgtgatg tttcagggag cttggtggta
caaaaactgc catgtgtcaa acctgaatgg tcgctacctc
agggggactc atggcagctt tgcaaatggc atcaactgga
agtcggggaa aggatacaat tatagctaca aggtgtcaga
gatgaaggtg cgacctgcct ag.

Human L-ficolin protein reference sequence translation of FCN2, isoform a, results in a 313 amino acid protein: meldravgvl gaatlllsfl gmawalqaad tcpevkmvgl egsdkltilr gcpglpgapg pkgeagtngk rgergppgpp gkagppgpng apgepqpclt gprtckdlld rghflsgwht iylpdcrplt vlcdmdtdgg gwtvfqrrvd gsvdfyrdwa tykqgfgsrl gefwlgndni haltaqgtse lrvdlvdfed nyqfakyrsf kvadeaekyn lylgafvegs agdsltfhnn qsfstkdqdn dlntgncavm fqgawwyknc hvsnlngryl rgthgsfang inwksgkgyn ysykvsemkv rpa (SEQ ID NO: 4).

Human H-ficolin nucleotide reference sequence, FCN3 CDS transcript variant 1 derived from consensus CDS 300.1/NCBI NM_{—}003665.2, comprises a 990 by sequence as follows:

(SEQ ID NO: 8)
atggatctac tgtggatcct gccctccctg tggcttctcc
tgcttggggg gcctgcctgcctgaagaccc aggaacaccc
cagctgccca ggacccaggg aactggaagc
cagcaaagttgtcctcctgc ccagttgtcc cggagctcca
ggaagtcctg gggagaaggg agccccaggtcctcaagggc
cacctggacc accaggcaag atgggcccca agggtgagcc
aggagatccagtgaacctgc tccggtgcca ggaaggcccc
agaaactgcc gggagctgtt gagccagggcgccaccttga
gcggctggta ccatctgtgc ctacctgagg gcagggccct
cccagtcttttgtgacatgg acaccgaggg gggcggctgg
ctggtgtttc agaggcgcca ggatggttctgtggatttct
tccgctcttg gtcctcctac agagcaggtt ttgggaacca
agagtctgaattctggctgg gaaatgagaa tttgcaccag
cttactctcc agggtaactg ggagctgcgggtagagctgg
aagactttaa tggtaaccgt actttcgccc actatgcgac
cttccgcctcctcggtgagg tagaccacta ccagctggca
ctgggcaagt tctcagaggg cactgcaggggattccctga
gcctccacag tgggaggccc tttaccacct atgacgctga
ccacgattcaagcaacagca actgtgcagt gattgtccac
ggtgcctggt ggtatgcatc ctgttaccga tcaaatctca
atggtcgcta tgcagtgtct gaggctgccg cccacaaata
tggcattgactgggcctcag gccgtggtgt gggccacccc
taccgcaggg ttcggatgat gcttcgatag.

Human H-ficolin protein reference sequence of 299 amino acids (translation of FCN3 transcript variant 1) is as follows: mdllwilpsl wllllggpac lktqehpscp gpreleaskv vllpscpgap gspgekgapgpqgppgppgk mgpkgepgdp vnllrcqegp rncrellsqg atlsgwyhlc 1pegralpvf cdmdtegggw lvfqrrqdgs vdffrswssy ragfgnqese fwlgnenlhq ltlqgnwelrveledfngnr tfahyatfrl lgevdhyqla lgkfsegtag dslslhsgrp fttydadhds snsncavivh gawwyascyr snlngryays eaaahkygid wasgrgvghp yrrvrmmlr (SEQ ID NO: 9).

The sequence of the L-ficolin MBL fusion proteins used in the experiments is set forth as follows:

L-FCN-MBL126, a 251 amino acid protein:

(SEQ ID NO: 5)
MELDRAVGVLGAATLLLSFLGMAWALQAADTCPEVKMVGLEGSDKLTILR
GCPGLPGAPGPKGEAGTNGKRGERGPPGPPGKAGPPGPNGAPGEPQPCLT
GPRTCKDLLDRGHFLSGWHTIYLPDCRPLTFSLGKQVGNKFFLTNGEIMT
FEKVKALCVKFQASVATPRNAAENGAIQNLIKEEAFLGITDEKTEGQFVD
LTGNRLTYTNWNEGEPNNAGSDEDCVLLLKNGQWNDVPCSTSHLAVCEFP
I.

L-FCN-MBL76, a 255 amino acid protein:

(SEQ ID NO: 6)
MELDRAVGVLGAATLLLSFLGMAWALQAADTCPEVKMVGLEGSDKLTILR
GCPGLPGAPGPKGEAGTNGKRGERGPPGPPGKLGPPGNPGPSGSPGPKGQ
KGDPGKSPDGDSSLAASERKALQTEMARIKKWLTFSLGKQVGNKFFLTNG
EIMTFEKVKALCVKFQASVATPRNAAENGAIQNLIKEEAFLGITDEKTEG
QFVDLTGNRLTYTNWNEGEPNNAGSDEDCVLLLKNGQWNDVPCSTSHLAV
CEFPI.

L-FCN-MBL64, a 254 amino acid protein:

(SEQ ID NO: 7)
MELDRAVGVLGAATLLLSFLGMAWALQAADTCPEVKMVGLEGSDKLTILR
GCPGLPGAPGPKGEAGTNGQGLRGLQGPPGKLGPPGNPGPSGSPGPKGQK
GDPGKSPDGDSSLAASERKALQTEMARIKKWLTFSLGKQVGNKFFLTNGE
IMTFEKVKALCVKFQASVATPRNAAENGAIQNLIKEEAFLGITDEKTEGQ
FVDLTGNRLTYTNWNEGEPNNAGSDEDCVLLLKNGQWNDVPCSTSHLAVC
EFPI.

In the above-identified sequences, the protein part indicated in bold indicates L-ficolin signal sequence; italics indicates L-ficolin component of fusion protein; and the remaining part is MBL component of fusion protein. The double-underlined K indicates a Lysine=MBL-associated serine protease (MASP) binding site.

The fusion protein numbers used herein refer to the corresponding amino acid from the MBL protein sequence (1-248). Accordingly, in the L-FCN-MBL126, the number 126 corresponds to the first amino acid (L) of the MBL component of this fusion protein (aa126-248 is the Carbohydrate Recognition Domain) L-ficolin component=aa1-128; in the L-FCN-MBL76, the number 76 corresponds to the first amino acid (L) of the MBL component of this fusion protein (aa126-248 is the Carbohydrate Recognition Domain) L-ficolin component=aa1-82; and in the L-FCN-MBL64, the number 64 corresponds to the first amino acid (Q) of the MBL component of this fusion protein (aa126-248 is the Carbohydrate Recognition Domain) L-ficolin component=aa1-64.

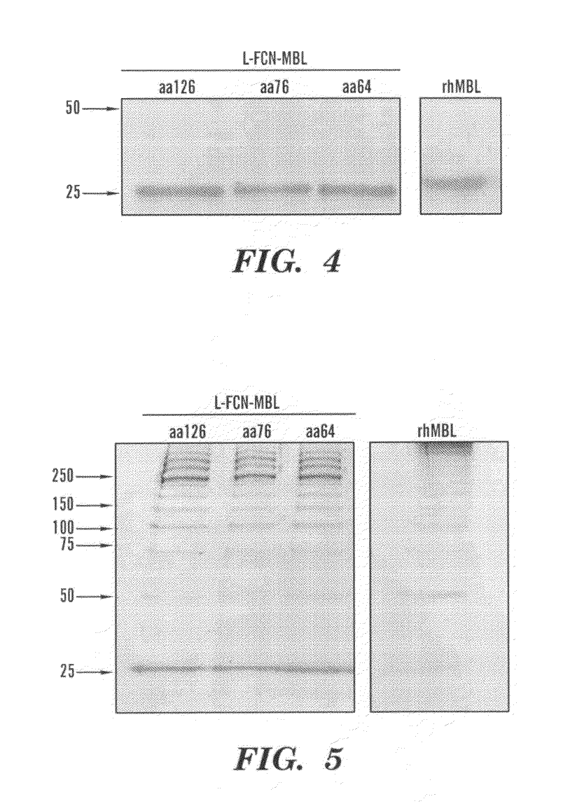

The chimeric FCN-MBL fusion proteins were expressed, purified and analyzed on a 4-20% gradient SDS-PAGE gel, which was stained with Imperial Blue after electrophoresis. The recombinant chimeric proteins were compared to the full length rhMBL. An aliquot of 450 ng of each of the recombinant proteins was separated under reducing conditions. On the gel, the chimeric proteins exhibited an apparent molecular weight of ˜30 kDa, slightly smaller than the full length rhMBL (FIG. 4). Only a single polypeptide was expressed for each construct.

Under non-reducing conditions (1200 ng purified protein), the recombinant chimeric proteins primarily form trimers and tetramers whereas rhMBL forms larger multimers (FIG. 5). The full-length rhMBL forms larger multimers than 3 chimeric proteins that comprise varying lengths of the carboxy-terminal of MBL and the amino-terminal of L-ficolin.

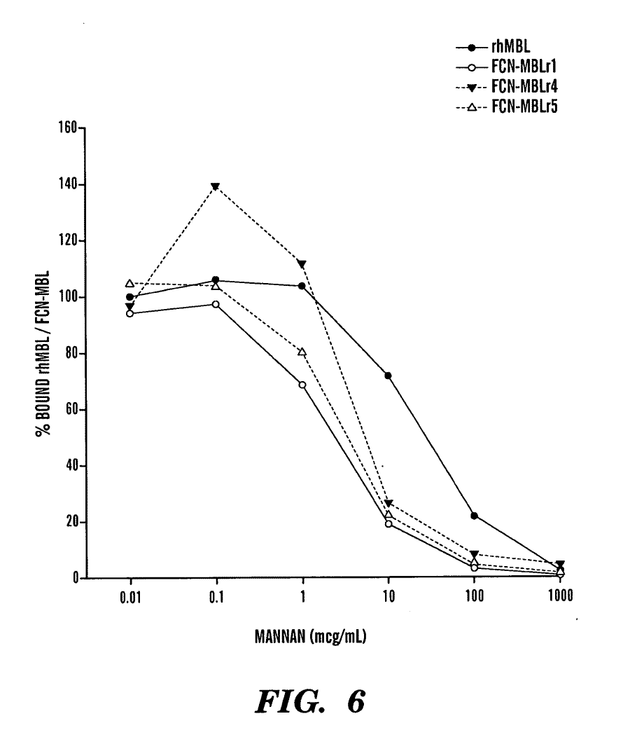

Binding of the rhMBL or any of the three chimeric FCN-MBL fusion proteins (10 mcg/mL in a 96-well ELISA format) to mannan was competed with mannan and then detected with anti-hMBL mAb (131-01). Binding to mannan was similar for all proteins except in a narrow range of concentrations (FIG. 6). The chimeric proteins bind a similar spectrum of carbohydrate ligands as demonstrated in the competitive ELISA assay.

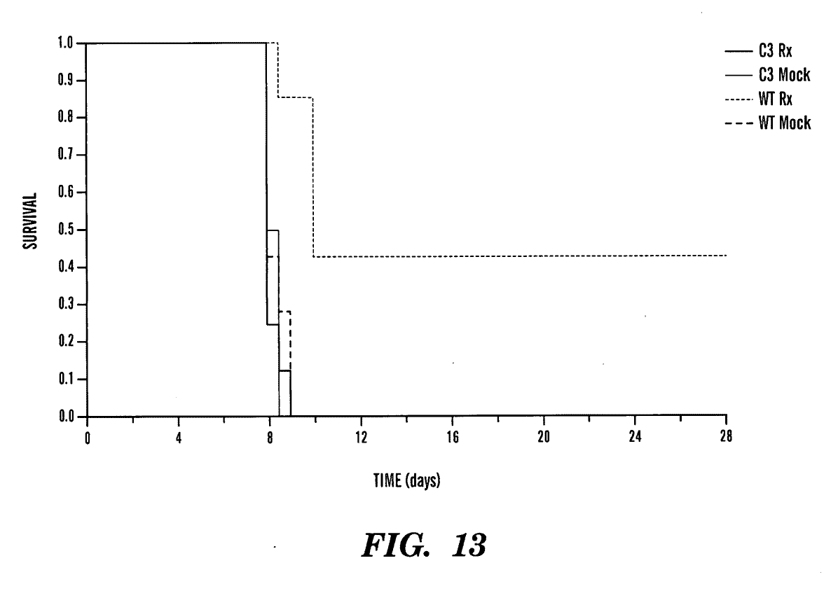

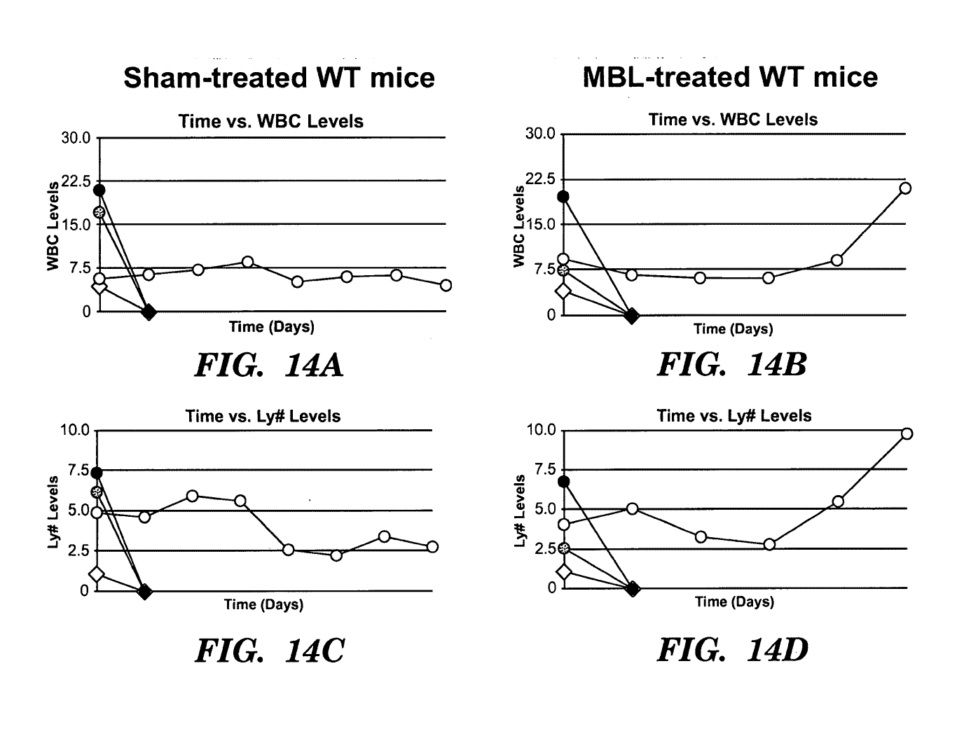

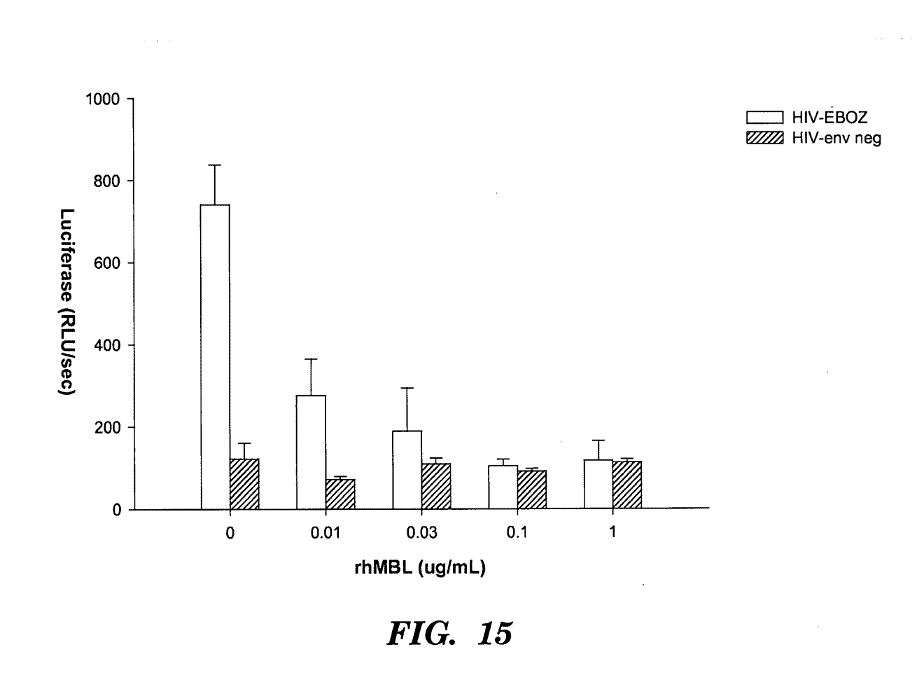

FIG. 13 shows a Kaplan Meier survival curve with a post-challenge analysis which demonstrated that recombinant human MBL-treated wild-type mice had a significant survival advantage: 40% survived compared to 100% mortality among wild-type and C3 knock-out mice treated with saline or rhMBL indicating that rhMBL provides protection but that the protection is dependent on C3. MBL treated mice survived significantly longer than mice not treated with MBL. EBOV was administered N 100pfu (plaque forming units) 3000xLD₅₀. WT (wildtype, C57B/6J mice) versus C3 knock out (KO). Recombinant MBL (rhMBL) was administered at 350 mcg IL 12 hors post challenge, then q12 h×10 days vs. sham Rx. *log rank, p<0.0004.

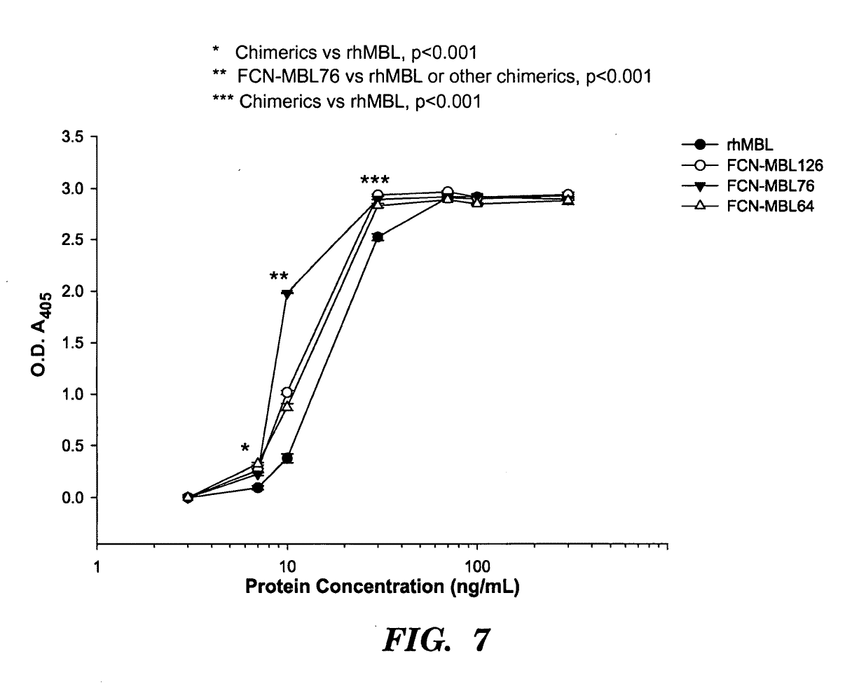

The rhMBL and the three chimeric FCN-MBL fusion proteins exhibited similar functional capacity to activate complement as determined by C4 deposition. FIG. 7 shows the C4 deposition assay that compared the capacity of rhMBL and the chimeric proteins to bind C4. This test indicates the complement activating activity of lectins. We showed that FCN-MBL76 has significantly greater C4 binding activity compared with rhMBL and the other chimerics.

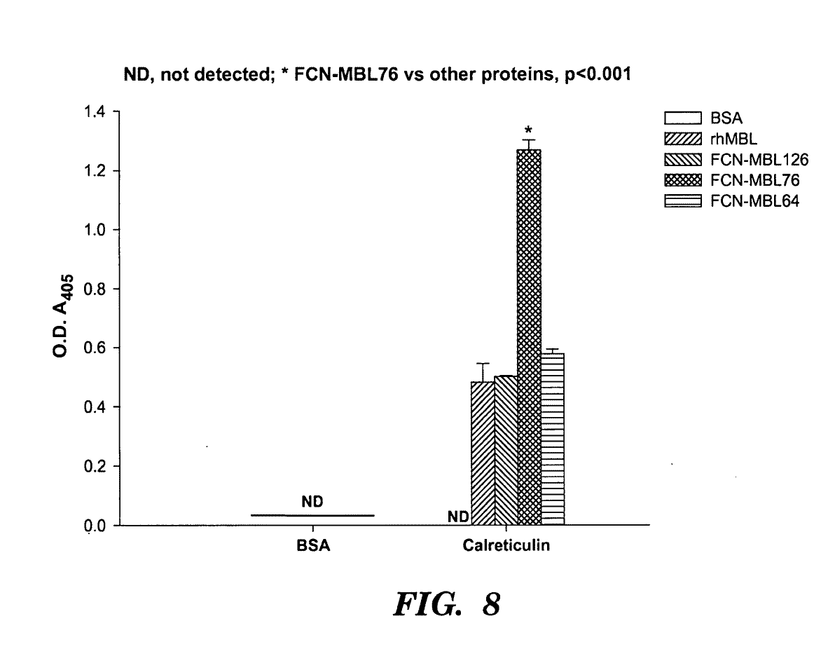

Calreticulin binding assay. The 96-well ELISA plate was coated with rhMBL or chimeric proteins (10 ug/mL), blocked with BSA and incubated with 5 ug/mL biotinylated human placental calreticulin that was measured at absorbance O.D. 405. FCN-MBL76 bound to human placental calreticulin significantly better than rhMBL or the other chimeric proteins. This likely has important implications for the relative functions of the proteins because calreticulin is the putative cellular receptor on phagocytes for native MBL and therefore, enhanced binding of the chimeric molecule results in improved pathogen clearance by opsonophagocytosis (FIG. 8).

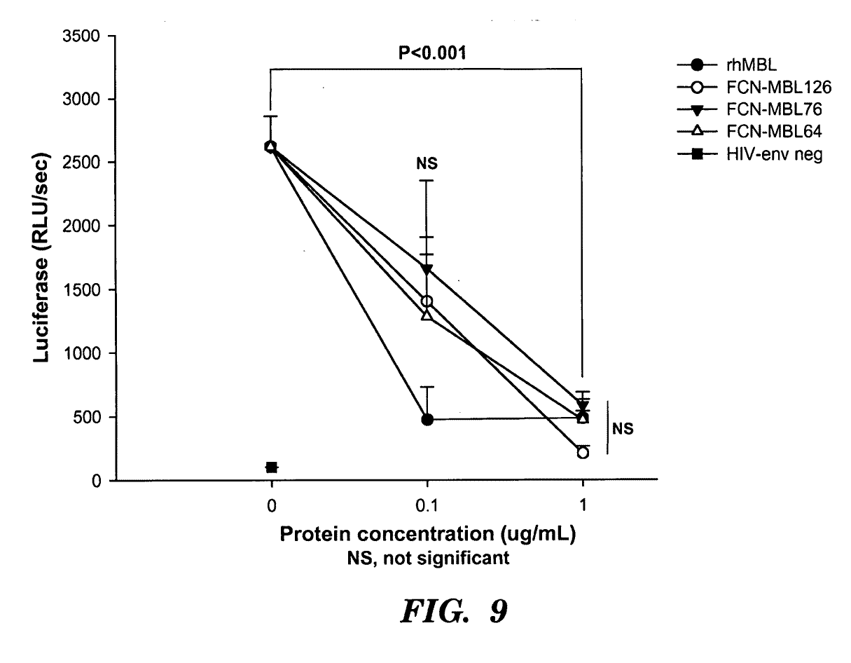

Inhibition assay using Hep G2 cells infected with lentivirus (HIV) pseudotyped with Ebola glycoprotein. Hep G2 cells at approximately 80% confluence in 96-well tissue culture plates were infected with HIV particles without an envelope (HIV-env neg; solid square) or with an envelope consisting of Ebola glycoprotein (other symbols). The virions encoded luciferase that was expressed only in infected cells and detected with a commercial luciferase assay. Before addition of viral particles to the cells, the viruses were preincubated with 0, 0.1 or 1 ug/mL of rhMBL or chimeric proteins in veronal-buffered saline with 5 mM CaCl2 for 1 hour at 37 C. Infection was achieved by spinoculation of cells at 1000 g×2 hrs. The viral protein mixture was replaced with EMEM culture media and incubated at 37 C for 40 hrs after which, the cells were lyzed and luciferase expression was quantified. rhMBL and the chimeric proteins inhibited viral infection to similar significant extents (1 ug/mL vs no protein, p<0.001) (FIG. 9).

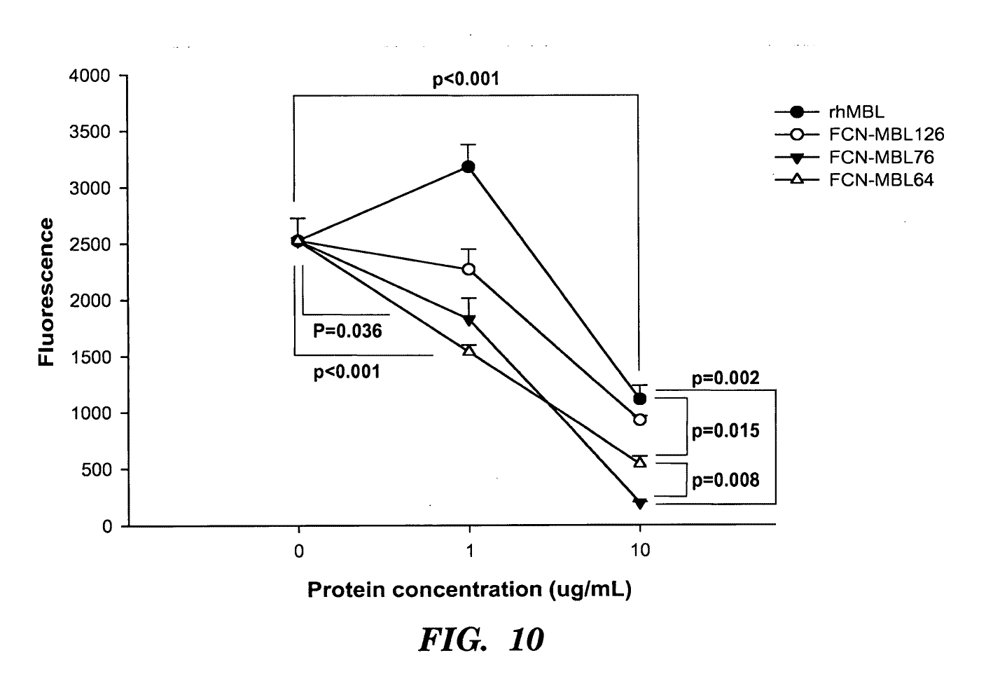

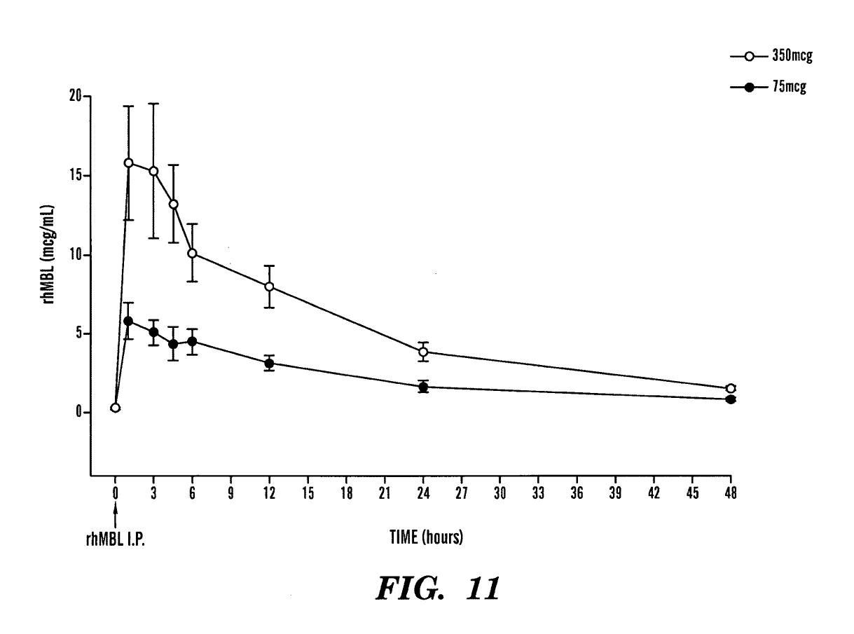

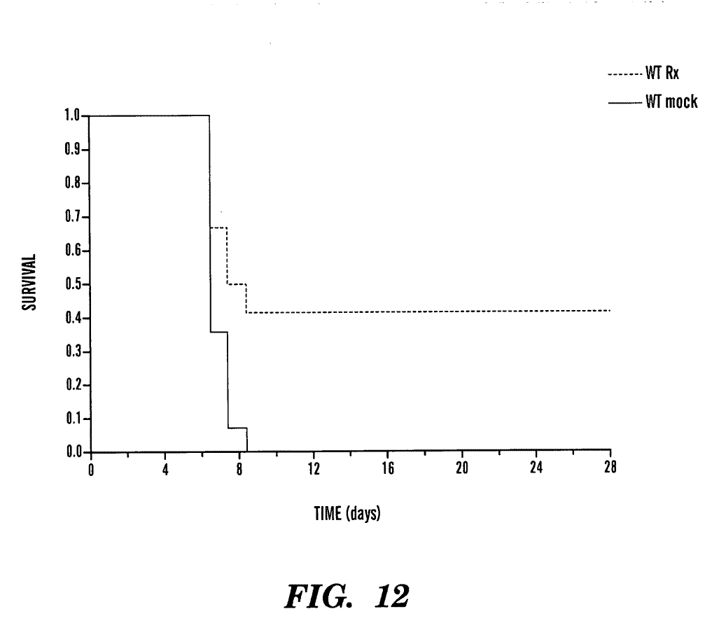

The pharmacokinetic modeling of rhMBL in immunocompetent C57B/6J mice revealed that doses of 75 mcg and 350 mcg doses produced Cmax of ˜5 mcg/mL and ˜15 mcg/mL, respectively and half-life of ˜11 hours at both doses (FIG. 10). A previous study showed that 75 mcg is the minimum dose of rhMBL required to activate complement in an MBL-deficient mouse model.

Using the higher dosage of 350 mcg/ml, the survival rate of mice infected with the EBOV Zaire virus was analyzed. Data is presented as the Kaplan Meier survival analyses. 350 mcg rhMBL was given immediately pre-challenge with EBOV Zaire and continued every 12 hrs×10 days. These group of mice had a 42% survival rate (log rank, p<0.008). When 350 mcg rhMBL was given 12 hours post-challenge with EBOV Zaire and continued every 12 hrs×10 days, the mice also had a 42% survival rate (log rank, p<0.002). Therefore, the MBL proteins have preventive/prophylactic function as well as treatment function against viral infections.

Accordingly, our data clearly demonstrate that rhMBL and chimeric FCN-MBL proteins activate complement (bind C4) to a similar extent. Since rhMBL has a half-life in mice of −11 hours, supraphysiologic doses of rhMBL administered in prophylactic and therapeutic regimens every 12 hours significantly reduced mortality by >40%.

The references cited herein and throughout the specification and examples are herein incorporated by reference in their entirety.

We claim:

1. A method for treatment and/or prevention of an infection in an individual comprising administering to said individual a supraphysiological amount of mannose-binding lectin (MBL) or ficolin-MBL fusion protein or fragments thereof and a pharmaceutically acceptable carrier.

2. The method of claim 1, wherein the infection is a viral infection.

3. The method of claim 1 wherein the infection is a bacterial infection.

4. The method of claim 2, wherein the viral infection is caused by an Ebola virus.

5. The method of any of the preceding claims, wherein the supraphysiological amount is an amount that results in blood concentrations of the MBL or the ficolin-MBL fusion protein at about 2-10 times the average physiological MBL serum concentration.

6. The method of any of the preceding claims, wherein the average physiological MBL serum concentration is about 2 μg/mL.

7. The method of any of the preceding claims, further comprising a step of selecting an individual who does not have a defective MBL function.

8. The method of any of the preceding claims, further comprising a step of selecting an individual who has normal MBL serum concentration.

9. Use of a supraphysiological amount of mannose-binding lectin (MBL) or ficolin-MBL fusion protein or fragments thereof in a medicament for treatment and/or prevention of an infection.

10. The use according to claim 9, wherein the infection is a viral infection.

11. The use according to claim 9, wherein the infection is a bacterial infection.

12. The use according to claim 10, wherein the viral infection is caused by an Ebola virus.

13. The use of any of the claims 9-12, wherein the supraphysiological amount is an amount that results in blood concentrations of the MBL or the ficolin-MBL fusion protein at about 2-10 times the average physiological MBL serum concentration.

14. The use of any of the claims 6-10, wherein the average physiological MBL serum concentration is about 2 μg/mL.
